(19) United States
(12) Patent Application Publication
(10) Pub. No.: US 2002/0177536 A1
(43) Pub. Date: Nov. 28, 2002

(54) Continuous preparation process for multiphase soaps

(76) Inventors: Steffen Sonnenberg, Holzminden (DE); Rolf-Gunther Schmidt, Holzminden (DE); Theodor Schmidt, Hoxter (DE); Jorg Edler, Holzminden (DE)

Correspondence Address: BAYER CORPORATION, PATENT DEPARTMENT, 100 BAYER ROAD, PITTSBURGH, PA, 15205, US

(21) Appl. No.: 09/939,299

(22) Filed: Aug. 24, 2001

(30) Foreign Application Priority Data
May 23, 2001 (DE) 10125132.7

(51) Int. Cl. A61K 7/50

(52) U.S. Cl. 510/141; 510/143; 510/146

(57) ABSTRACT

In a continuous process for the preparation of multiphase soaps, in which stable multiphase soaps are obtained by suitable cutting and stamping of soap masses in various sequences, the individual phases of the multiphase soap are readily visible when viewed from above and from the side.

### FIELD OF THE INVENTION

The invention relates to continuous preparation processes for multiphase soaps in which the individual phases of the soap are readily visible when viewed from above and from the side.

### BACKGROUND OF THE INVENTION

DE 100 46 469.6-41 describes multiphase soaps in which the individual phases of the soap are readily visible when viewed from above and from the side, the manual preparation thereof and the application properties thereof. A continuous preparation process for these multiphase soaps is not described.

U.S. Pat. No. 5,198,140 describes the continuous preparation of an interlocked soap with increased strength. The simultaneous extrusion of two soap slugs through an extrusion head results in a soap slug interlocked in the horizontal direction. A subsequent single cut of the soap slug into billets and subsequent stamping produces horizontally divided soap bars.

EP A 0 366 209 describes the preparation of horizontal multiphase soaps by a casting process. Casting processes are only suitable for the preparation of small bar numbers but not for the production of soaps for the mass consumer market.

EP A 0 594 077 describes the preparation of spiral-shaped multiphase soaps which are prepared using a special compression head following radial rotation of the soap slug. Particularly in cases where different soap bases are used, the stability of the type of soap is limited in its application by the many phase boundaries.

DE-A 1 924 980 describes a process for the preparation of a multiphase soap with one or more sheaths which surround a core.

Analogous statements also apply to soaps prepared in accordance with JP 62/48799. Here, a multilayer circular slug is produced.

Also known are soaps in which a vertical cut in the transverse or in the longitudinal direction of the soap which separates the two soap phases (e.g. JP 1/247499). In this type of soap, both phases are visible at the same time. However, the vertical type of soap, upon use by the consumer and during continuing storage, displays the decisive disadvantage of lower stability of the overall soap bar. Because of the small and straight contact surfaces, a vertically cut soap may break apart even as a result of the soap simply falling to the ground.

EP-A 0 545 716 describes the preparation of a multidimensionally curved two-phase soap. By using the casting process, a two-phase soap is produced which is not suitable for the mass consumer market due to the costly preparation. In the case of this type of soap, no pressure is exerted subsequently in the form of a stamping operation.

Furthermore, marbled soaps are also known (DE-A 2 455 982, DE-A 2 431 048 and DE-A 1 953 916). Here, differently colored soap phases are mixed intensively with one another using special pressing cylinders or screw presses, so that a marbled effect arises. Furthermore, the color can also be injected into the soap stream during preparation of the soap. These soaps contain one phase.

A further multicolored single-phase soap is described in U.S. Pat. No. 4,435,310. Here, by injecting color during the preparation of the soap slugs and by manually rotating the extruder head, a multicolored sinusoidal soap is obtained from one billet.

Production of Toilet Soap (D. Osteroth, pp. 76-77, ISBN 3-921956-55-2) describes the cutting of soap slugs using a cutting machine whose blade is attached to a continuous conveyor belt. As a result of the continuous movement of the soap slug, the individual blades are driven into the soap slug and slice it. Here, the cut is perpendicular to the soap slug at an angle of 90°. The individual soap sections are then pressed in a press to give soap bars. With this design of the cutting machine, only an angle of 90° for the blades can be set through the positioning of the conveyor belt. Other cutting angles are not possible.

### SUMMARY OF THE INVENTION

An object of the present invention is the development of a continuous preparation process for multiphase soaps of the transverse and longitudinal type (FIGS. 1A, 1B and 3A, 3B) in which the individual phases of the soap are readily visible when viewed from above and from the side. Upon use, the multiphase soaps should have a stability comparable with that of a single-phase soap.

The invention, therefore, provides a continuous preparation process for multiphase soaps of the transverse and longitudinal type, containing two or more separately extruded phases, characterized in that the mutual cambering which arises as a result of the stamping of the multiphase soap slugs effects high stability of the finished soaps and the individual separate phases of the resulting multiphase soaps are readily visible when viewed from above and from the side.

In addition, a plurality of embodiments of the process according to the present invention have been developed.

The multiphase soaps from the preparation process according to the present invention exhibit superproportional strength which corresponds almost to the stability of a single-phase soap and which does not break in daily use.

### BRIEF DESCRIPTION OF THE DRAWINGS

FIG. 1A shows the top view of multiphase soap of the present invention.

FIG. 1B shows the perspective view of multiphase soap of the present invention.

FIG. 2A shows the top view of the cambering of the two phases of the soap of the present invention.

FIG. 2B shows the perspective view of the cambering of the two phases of the soap of the present invention.

FIG. 3A shows the top view of the longitudinal type of the soap of the present invention.

FIG. 3B shows the perspective view of the longitudinal type of the soap of the present invention.

FIG. 4A shows the top view of the multiphase soap with different cutting angles.

FIG. 4B shows the side view of the multiphase soap with different cutting angles.

FIG. 5 shows a schematic diagram of the process according to process A for the transverse type.

FIG. 6 shows a diagram of the cutting machine with conveyor belts according to FIG. 5.

FIG. 7 shows diagrams of the dies for targeted pinched edge removal and pinched edge separation for the transverse type.

FIG. 8 shows the schematic diagram for the preparation process according to process B for the longitudinal type and the transverse type.

FIG. 9A shows a diagram of the extrusion system for process B.

FIG. 9B shows a diagram of the extrusion system for process C.

FIG. 10 shows a diagram of the die with targeted pinch edge removal and pinched edge separation for the longitudinal type.

### DETAILED DESCRIPTION OF THE INVENTION

Preference is given to continuous preparation processes for multiphase soaps in which each phase is visible in the vertical, longitudinal and transverse projection to at least 15% based on the total projected area.

More preference is given to continuous preparation processes for multiphase soaps in which each phase is visible in the vertical, longitudinal and transverse projection to at least 20% based on the total projected area.

In a further embodiment of the preparation process according to the present invention, the phase areas which border one another in the resulting multiphase soaps are cut diagonally and cambered towards one another. The cambering is achieved during the continuous preparation by using pressure. Multiphase soaps with cambered interfaces have particular stability.

In the continuous preparation process according to the present invention for multiphase soaps, preference is given to using phases which have a different composition.

For the purposes of the present invention, soap phases contain soap bases and further additives or ingredients.

The soap bases for the preparation process for multiphase soaps are known per se (Soaps and Detergents, Luis Spitz, ISBN 0-935315-72-1 and Production of Toilet Soap, D. Osteroth, ISBN 3-921956-55-2). For example, soap bases such as alkali metal soaps containing animal and/or vegetable substances, syndets containing synthetic surfactants or combinations of the two can be used for the multiphase soaps.

The soap mass can include, as further ingredients, for example, perfume oils, cosmetic ingredients, dyes and further additives.

Examples of fragrances in the perfume oils for the multiphase soaps according to the present invention are given, for example, in S. Arctander, Perfume and Flavor Materials, Vol. I and II, Montclair, N.J., 1969, published privately or K. Bauer, D. Garbe and H. Surburg, Common Fragrance and Flavor Materials, 3rd Ed., Wiley-VCH, Weinheim 1997.

The perfume oils are generally added to the soap base in an amount of from 0.05 to 5% by weight, preferably from 0.1 to 2.5% by weight, most preferably from 0.2 to 1.5% by weight, based on the soap base.

The perfume oils may be added in liquid form, neat or diluted with a solvent for perfuming the soap base. Suitable solvents for this purpose are, for example, ethanol, isopropanol, diethylene glycol monoethyl ether, glycerol, propylene glycol, 1,2-butylene glycol, dipropylene glycol, diethyl phthalate, triethyl citrate, isopropyl myristate etc.

In addition, the perfume oils for the multiphase soaps according to the present invention can be adsorbed on a carrier which serves both to finely distribute the fragrances within the product and to release them in a controlled manner during use. Such carriers can be porous inorganic materials, such as light sulfate, silica gels, zeolites, gypsums, clays, clay granulates, gas concrete etc. or organic materials such as woods and cellulose-based substances.

The perfume oils for the multiphase soaps in the preparation process according to the present invention can also be in microencapsulated form, spray-dried form, in the form of inclusion complexes or in the form of extrusion products and can be added in this form to the soap base to be perfumed.

The properties of the perfume oils modified in this way may, where appropriate, be further optimized with regard to a more targeted fragrance release by “coating” with suitable materials, for which purpose preference is given to using wax-like polymers such as, for example, polyvinyl alcohol.

Cosmetic ingredients for the multiphase soaps according to the present invention are known per se (Soaps and Detergents, Luis Spitz, ISBN 0-935315-72-1 and Production of Toilet Soap, D. Osteroth, ISBN 3-921956-55-2). For example, the following cosmetic ingredients may be mentioned:

Cooling active ingredients, such as e.g. menthol and menthol derivatives, warming active ingredients, such as, for example, capsaicin, UV filters, such as, for example, Neo Heliopane® (A registered trademark of Haarmann & Reimer GmbH) to protect against discoloration of the soap or to protect against solar irradiation on the skin, vitamins, such as, for example, vitamins A and E for vitalizing the skin, vegetable waxes and oils, such as, for example, cocoa butter, almond oil, avocado oil and jojoba oil, for improving the feel on the skin, plant extracts, moisturizers, minerals, antidandruff active ingredients, such as, for example, Crinipan™, active ingredients, such as, for example, deodorizing active ingredients sodium carbonate, triclosan and triclocarban.

The following additives are also known: dyes, such as, for example, titanium dioxide, the addition of stabilizers, such as, for example, DTPA and EDTA, the addition of antioxidants, such as BHT, the addition of filling materials, such as, for example, starch and cellulose, the addition of hardeners, such as, for example, sodium chloride and sodium sulfate.

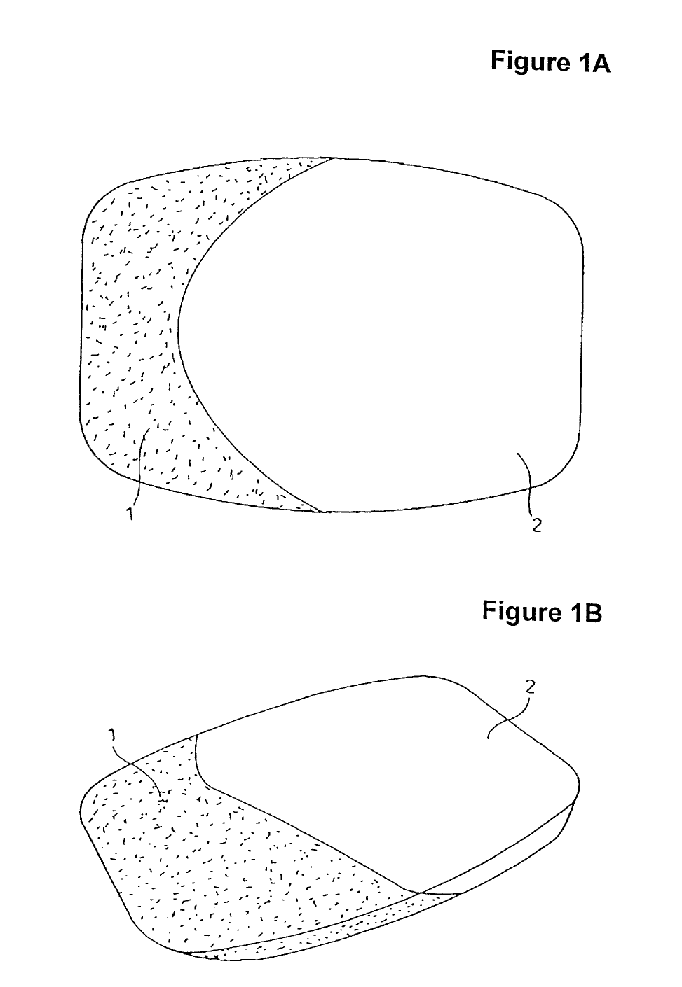

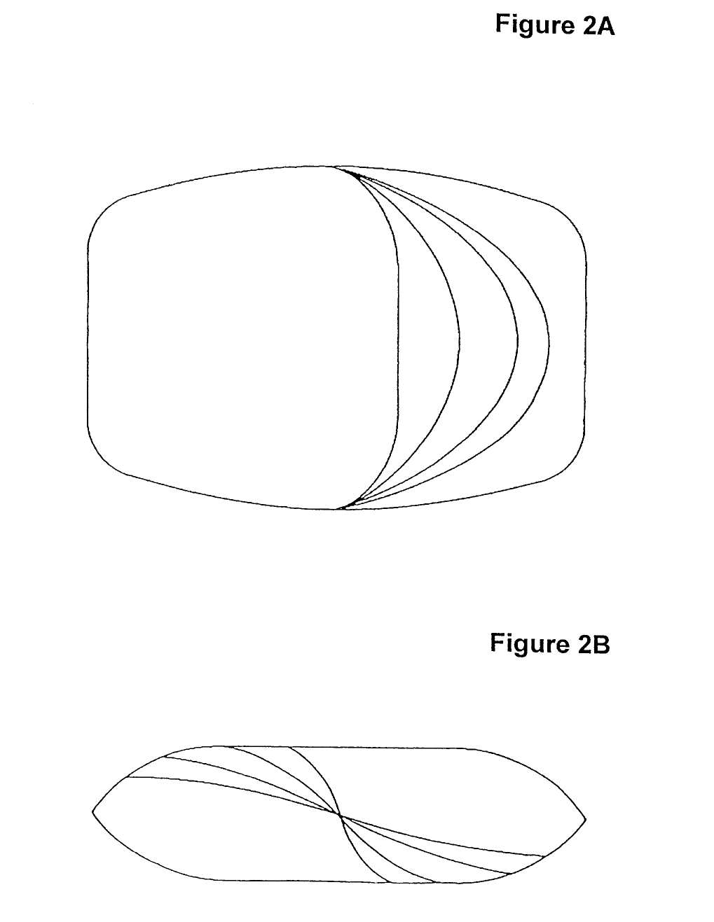

As a preferred embodiment of the process according to the present invention, we have found a process A1 for the continuous preparation of multiphase soaps of the transverse type (FIGS. 1A, 1B) which is characterized in that, after the milling, homogenization or kneading, two or more simultaneously extruded soap slugs comprising different soap phases are each cut with cuts diagonal to the soap slug at an angle of from 20° to 70°, the billets are mutually joined to give one soap slug, the soap slug is cut perpendicularly at an angle of 90°, and then the diagonal points of intersection are joined under pressure so that cambering arises at the points of intersection (FIGS. 2A, 2B).

Preferably, in the process A1, the diagonal cut can be made first, then the 90° cut and then the joining of the soaps prior to stamping (process A2).

Also preferably, in the process A1, the 90° cut can also be made first, then the diagonal cut and then the joining of the soaps prior to stamping (process A3).

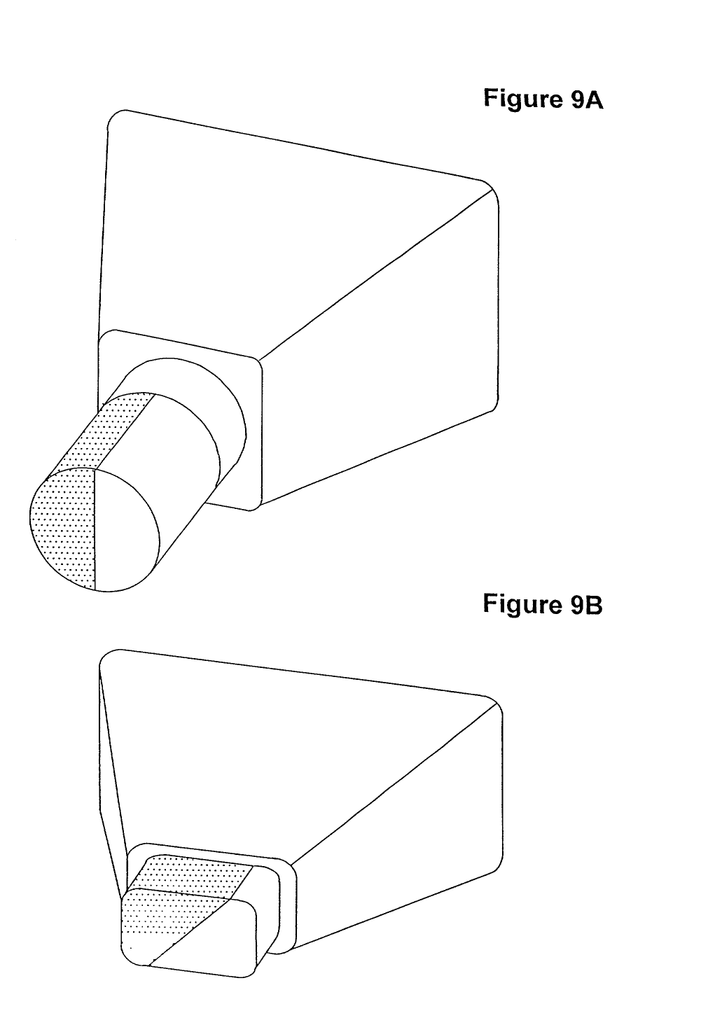

In addition, we have found a process B for the continuous preparation of multiphase soaps of the transverse type (FIGS. 1A, 1B), which is characterized in that, after the milling, homogenization or kneading of the soap phases, two soap slugs containing different soap phases are coextruded with a contact surface through a mouthpiece (FIGS. 9A, 9B), the contact surfaces being rotated by an angle of from 20° to 70° about the longitudinal axis from the perpendicular, then the two joined soap slugs are cut at an angle of 90°, the resulting soap slugs are rotated by 90° about the horizontal and then the contact surfaces of the individual soap phases are joined by stamping under pressure so that cambering arises at the points of intersection (FIGS. 2A, 2B).

In addition, it is preferred if, after the cutting of the soap slug, the subsequent joining under pressure is carried out directly by suitably arranging the die and without rotation of the soap slugs.

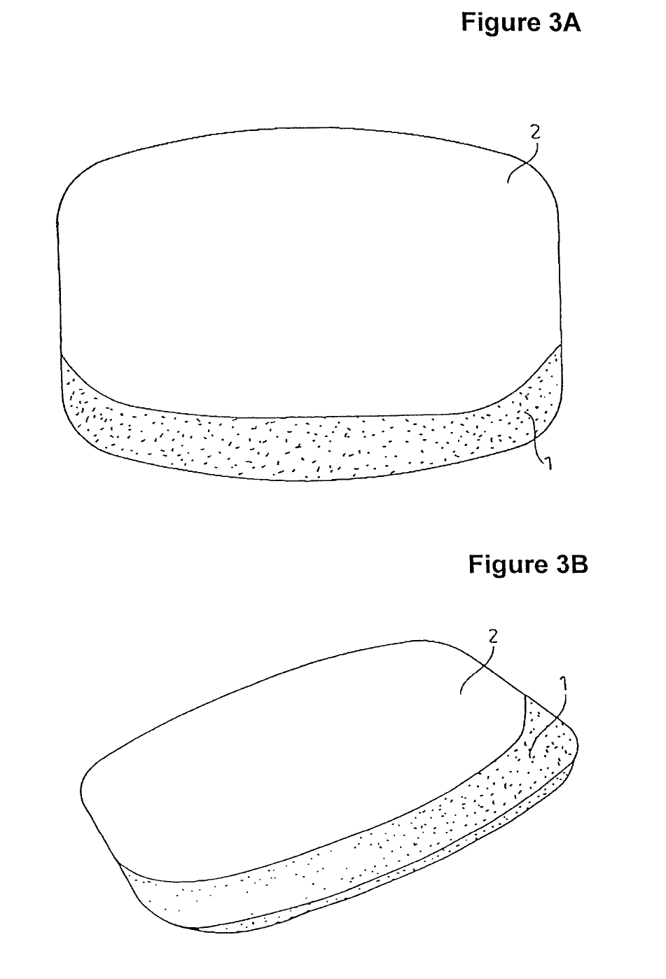

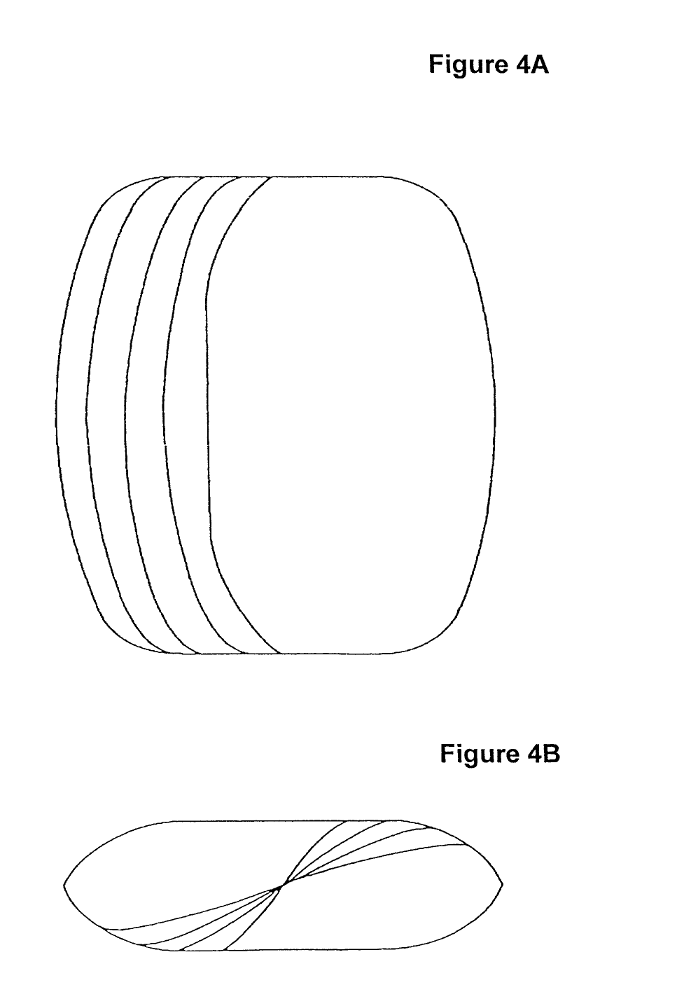

In addition, we have found a process C1 for the continuous preparation of multiphase soaps of the longitudinal type (FIGS. 3A, 3B), which is characterized in that, after the milling, homogenization or kneading of the soap phases, two soap slugs containing different soap phases are coextruded with vertical contact surfaces through a mouthpiece, the two joined soap slugs are cut at an angle of 90°, the billets are rotated by an angle of from 20° to 70° about the longitudinal axis from the perpendicular and then the contact surfaces of the individual soap phases are joined under pressure so that cambering arises at the points of intersection (FIGS. 4A, 4B).

Alternatively, in process C1, it is also possible, after the milling, homogenization or kneading of the soap phases, to carry out the coextrusion of the two individual soap slugs rotated by an angle of from 20° to 70° about the longitudinal axis from the perpendicular. The soap slug is then cut at an angle of 90° and then the contact surfaces of the individual soap phases are joined under pressure, so that cambering arises at the points of intersection (process C2).

The multiphase soaps prepared by the process according to the invention are described, in terms of their design, in DE 100 46 469.6-41 and can be illustrated by reference to FIGS. 1A, 1B and 3A, 3B by way of example but not in a limiting manner for a rounded standard soap shape with a length of 7.4 cm, a height of 1.8 cm and a width of 5.4 cm: FIGS. 1A, 1B and 3A, 3B show the multiphase soap in perspective view and in top view. The different phases are in each case, designated 1 and 2. FIGS. 2A, 2B and 4A, 4B also represent the cambering of the two phases in top view and side view.

The multiphase soaps of the transverse and longitudinal type can be prepared by the continuous processes A, B and C according to the invention in an effective and favorable manner in large bar numbers and have high stability.

Preparation Processes

The preparation of soap masses is known (Soaps and Detergents, Luis Spitz, ISBN 0-935315-72-1 and Production of Toilet Soap, D. Osteroth, ISBN 3-921956-55-2).

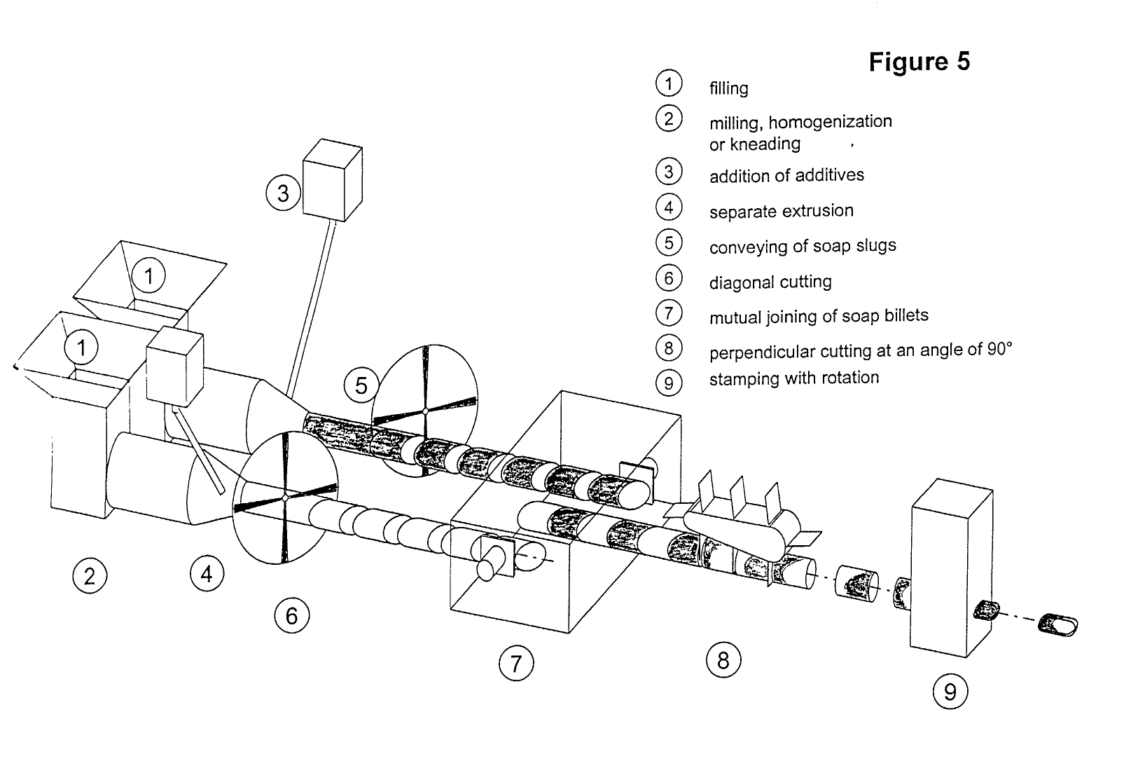

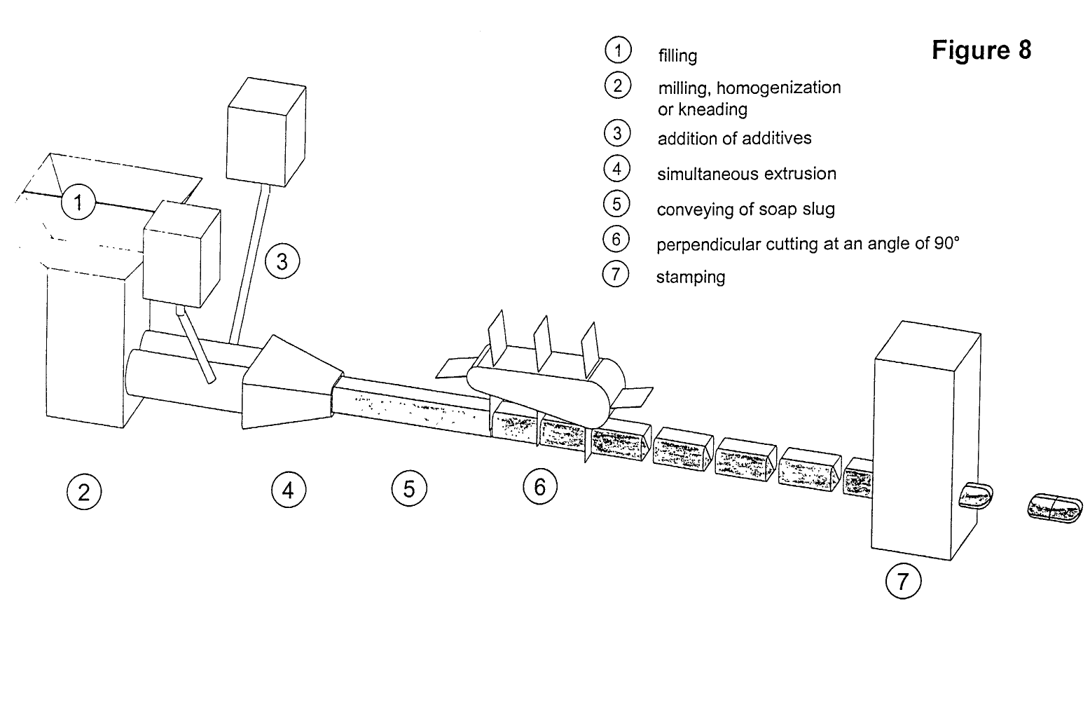

The multiphase soaps are prepared as described in process A by FIG. 5 and in processes B and C by FIG. 8 as an example: first, the soap bases (FIGS. 5 and 8, step 1) are admixed with the above-described additives such as perfume oil, cosmetic ingredients, dyes, stabilizers and further additives, and then milled and/or homogenized using various rollers or kneaders (FIGS. 5 and 8, step 2).

Process A for the transverse type: after the homogenization, the two different soap masses, which can differ by virtue of the composition of the soap mass itself, or by virtue of at least one of the above-mentioned additives, are simultaneously extruded (FIG. 5, step 4). Here, various jacket temperatures can be used depending on the extruder used or the screw press used and the soap masses.

Alternatively, various liquid additives can also be added to the soap mass at a later point in time by metered addition via nozzles (FIG. 5, step 3).

The resulting soap slugs are conveyed in parallel on two or more conveyor belts (FIG. 5, step 5).

Then, in contrast to the customary cutting angle of 90°, the simultaneously obtained soap slugs are cut corresponding to the subsequent soap shape and the design type firstly diagonally at an angle of from 20° to 70° (FIG. 5, step 6). The diagonal cut of both soap slugs is performed by two or more separate cutting devices or by a combined cutting device.

The soap slugs are diagonally divided by a blade which moves up and down or forwards and backwards and which is guided diagonally perpendicularly or diagonally horizontally relative to the soap slug.

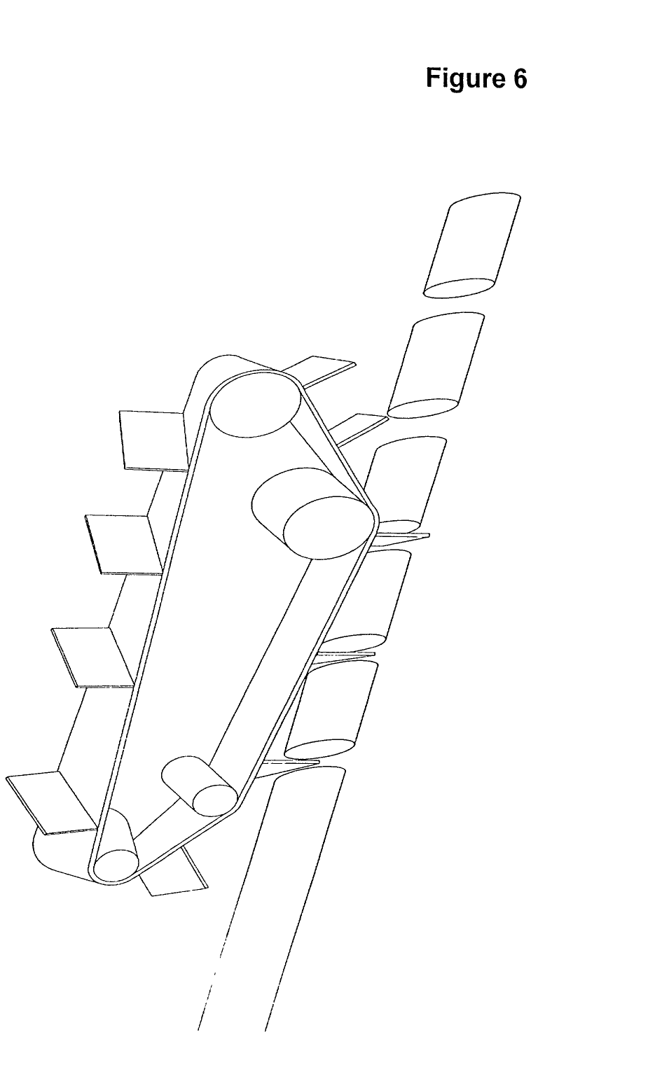

The diagonal cut of the soap slug is preferably carried out using a continuous conveyor belt on which diagonally attached blades are located (FIG. 6). The conveyor belts travel on two oppositely positioned cones which ensure even and trouble-free rotation of the blades. The angles of the two cones are determined here by the angling of the blades relative to the soap slug. As a result of the cutting, soap billets in the shape of a parallelogram are obtained.

To cut the soap slug, the conveyor belts are passed over appropriately positioned deflection rollers. In this connection, the precise cut of the blades is ensured by the reliable conveyance on the conveyor belt and on the rotating cones.

The cutting angle of the blades relative to the soap slug can be changed in a flexible manner via the positioning of the blades on the conveyor belt and via the angle of the rotating cones. This special technical feature permits the clean cutting of soap billets into large bar numbers.

Propulsion of the blades can either be carried out by the propulsion of the soap slug or by a direct drive. In the case of a direct drive, the speed of the blades is synchronized with the propulsion of the soap slug.

In a preferred embodiment, the soap slug is cut by blades located on a roller. This ensures a clean cut as a result of the size and the position relative to the soap slug.

It is preferred if the soap slug is cut by blades attached to a rotating disc (FIG. 5, step 6). The disc is arranged obliquely relative to the direction of transportation of the soap slug in order to obtain the oblique cut. The speed of rotation is synchronized with the speed of the conveyor belt in order to obtain billets of the desired length.

In addition, it is preferred if the soap slug is divided by targeted diagonal stamping, by cutting with a wire or by other methods. In this connection, reliable cutting is ensured by the suitable positioning of the soap slug e.g. by concave trays.

In all of the processes A used, the soap slug is divided diagonally relative to the axis of the soap slug at an angle of from 20° to 70°, preferably 25° to 60° and most preferably 30° to 50°.

The alternating bringing together of the billets of the two individual soap slugs is preferably carried out using material-stream combiners (FIG. 5, section 7), in which the diagonal cut surfaces of the individual sections are guided precisely to one another and optionally slightly pressed.

Preference is given to the bringing together of the billets by displacement e.g. using vacuum suction bells. Here, either individual or simultaneously two or more billets are lifted up from the first two conveyor belts and placed onto a third conveyor belt. This conveyor belt, which is synchronized with the first two conveyor belts, travels at approximately twice the speed. The third conveyor belt can be flat or slightly cambered and preferably has indentations or edges within which the billets are held.

It is also preferable to bring together the individual billets using material-stream diverters, e.g. metal paddles, which are at a plane angle relative to the conveyor belt and divert the billets onto another conveyor belt. In this connection, the new conveyor belt must travel at at least twice the speed as the first two conveyor belts. As a result of the doubled speed of the third conveyor belt, a gap between the billets of the first conveyor belt is created, into which the billets of the second conveyor belt are then inserted. The third conveyor belt can be flat or slightly cambered and preferably has indentations or edges within which the billets are held.

To improve the adhesion of the individual billets, the cut surfaces can also be wetted with a fixative such as, for example, an adhesive or with warm or liquid soap mass. After the joining together and the setting or cooling, the join becomes strong. The setting or cooling can be influenced by suitable temperature control.

If an adhesive is used, it must be ensured that no residues arise as the soap is worn away by, for example, using water-soluble adhesives.

Then, the newly combined soap slug is cut a second time at the customary angle of 90° corresponding to the length of the subsequent soap bar (FIG. 5, step 8), the cut preferably being in the middle of the cuts already present. Alternatively, the cut can be offset from the centre. At the end of this process, this will give a soap bar with differently sized individual soap phases.

It is also preferred if the individual soap slugs are first cut, diagonally, at an angle of from 20° to 70°, then cut again at an angle of 90° and then combined to give one soap slug or to give the desired cylindrical soap slugs.

It is also preferred, if the individual soap slugs are cut, first at an angle of 90°, then again diagonally at an angle of from 20° to 70° and are then combined to give one soap slug or to give the desired cylindrical soap slugs.

The newly formed cylindrical soap slugs, which contain different soap phases, are then pressed into the final shape by horizontal or vertical stamping or pressing (FIG. 5, step 9). Alternatively, the cylindrical soap billet can be rotated and then stamped. Prior to stamping, the precise positioning of the two billets and of the overall soap slug in the die is important. This can be ensured, for example, by the suitable choice of guides and holders, which press the sections together, both before and also during the stamping operation.

The stamping of the cut and combined soap slugs to give the soap bars is usually carried out individually in rotating disc presses or in greater numbers in so-called flashstamping systems (Soaps and Detergents, Luis Spitz, pp. 193-204, ISBN 0-935315-72-1).

The stamping operation is carried out with a varying pressing weight or pressing force, depending on the type of soap stamping machine used. Various temperatures are used during the stamping operation, corresponding to the nature and the composition of the soap mass.

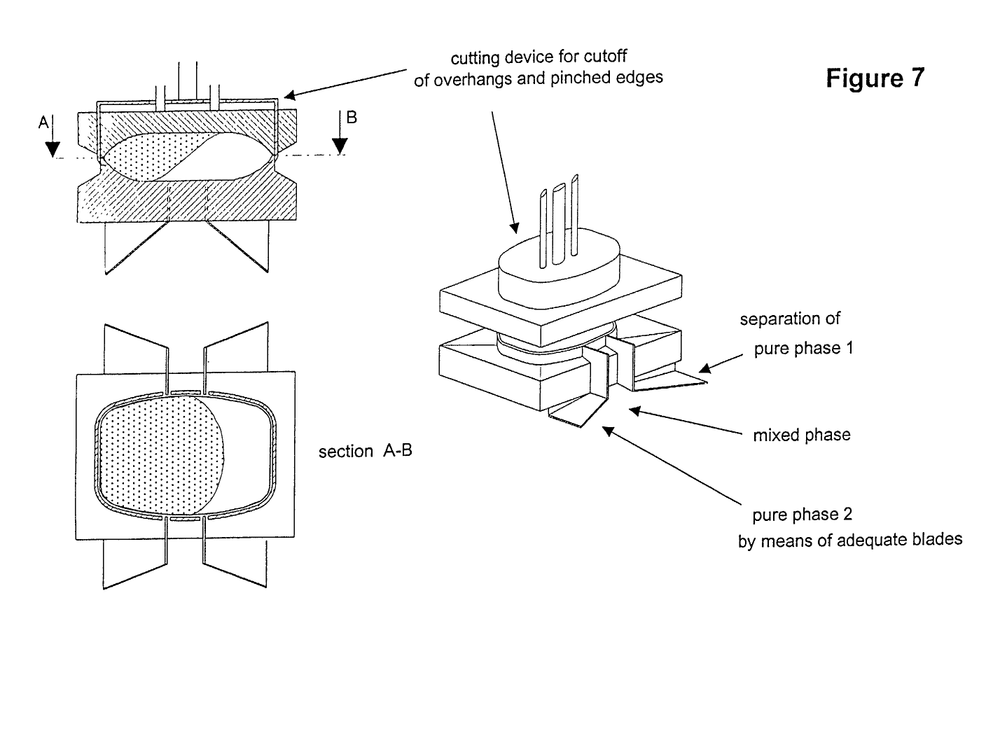

The overhangs and pinched edges which arise during stamping are separated from the soap bar using blades or guides (FIG. 7) during the actual stamping operation. Here, by virtue of the suitable arrangement of the blades, both the two pure soap masses and also a mixed phase is separated off. The two pure soap phases are then returned to the preparation process. For the die according to FIG. 7, a mixed phase of about 1%, based on the total soap mass used in the first substep, arises.

This mixed phase can either be processed to give a separate soap or, in the case of a two-colored soap, be colored and likewise reused.

Alternatively, two or more diagonal soap sections obtained from the first diagonal cut can also be pressed individually in a horizontal press following the mutual joining. As a result, stamping takes place continuously from the soap slug. The protruding cut edge of the two diagonally cut bars is then likewise separated and can be returned to the process.

Finally, the resulting soap bars are cooled to normal temperature in cooling systems.

Process B for the transverse type: after the homogenization, the two different soap masses, which can differ by virtue of the composition of the soap mass itself or else by virtue of at least one of the abovementioned additives, are extruded by coextrusion (FIG. 8, step 4) to give a soap slug.

Alternatively, various liquid additives can also be added to the soap mass by metered addition via nozzles at a later point in time (FIG. 8, step 3).

First, the two soap masses are preshaped in individual, e.g. semicircular, extrusion pipes to give partial soap slugs, brought together in a common e.g. circular, extrusion pipe and then extruded through a mouthpiece (FIGS. 9A, 9B). This produces a longitudinally divided soap slug in which the soap phases have already been joined together as a result of the coextrusion.

By rotating the semicircular extrusion pipes of the partial soap slugs or by rotating the separating wall between the partial soap slugs from the perpendicular by an angle between 20° to 70° with respect to the longitudinal axis, a soap slug rotated from the perpendicular with respect to the two soap phases is obtained (FIGS. 9A, 9B).

It is preferable if the separating wall between the two individual extrusion pipes is a straight wall. It is also preferred if the separating wall has a wave shape matched to the subsequent soap shape, or an irregular shape. It is also preferred if the separating wall is in itself rotated and a rotation of the soap slugs from the perpendicular is achieved as a result.

It is preferred, if the individual extrusion pipes are semicircular, and the coextrusion pipe and the mouthpiece are circular in their geometric shape. Alternatively, the individual extrusion pipes may be semi-oval and the coextrusion pipe and the mouthpiece may be oval. It is further preferred if the individual extrusion pipes are square or rectangular and the coextrusion pipe and the mouthpiece are rectangular or square. Further combinations of circular, semicircular and angular extrusion pipes are possible.

The width and height of the mouthpiece is determined by the design of the finished soap bar. In a particular embodiment, the width of the mouthpiece corresponds to the length of the finished soap bar. Depending on the design of the finished soaps, the width of the mouthpiece is larger or smaller than the length of the die. In general, the volume of the soap slug must be greater than the volume of the die.

During the extrusion, various jacket temperatures can be used, depending on the extruder used or the screw press used and the soap masses. The extruded soap slug then contains two partial slugs which are joined to one another at a diagonal contact surface.

The soap slug is then cut using methods known per se at an angle of 90° corresponding to the size and shape of the finished soap bar. The length of the section is determined by the design of the finished soap bar. In a preferred embodiment, the length of the section corresponds to the width of the finished soap bar.

Thereafter, the soap slugs are rotated by 90° about the horizontal and converted into the final shape by stamping or pressing. The stamping of the soap slugs to give the soap bars is usually carried out individually in rotating disc presses or in greater numbers in so-called flashstamping systems (Soaps and Detergents, Luis Spitz, pp. 193-204, ISBN 0-935315-72-1).

The stamping operation is carried out with a varying pressing weight or pressing force depending on the type of soap stamping machine used. Various temperatures are used during the stamping operation, corresponding to the nature and the composition of the soap mass.

Preferably, the stamping or pressing of the soap bar can also take place without prior rotation of the soap slug. In this case, the die in the press is then conveyed at a right angle relative to the soap billet.

The overhangs and pinched edges which arise during stamping are separated from the soap bar using blades or guides (FIG. 7) during the actual stamping operation. Here, by virtue of a suitable arrangement of the blades, both the two pure soap masses and also a mixed phase is separated off. The two pure soap phases are then returned to the preparation process. For the die according to FIG. 7, a mixed phase of about 1%, based on the total soap mass used in the first substep, arises.

This mixed phase can either be processed to give a separate soap or can, in the case of a two-colored soap, be colored and likewise reused.

Finally, the resulting soap bars are cooled to normal temperature in cooling systems.

Process C for the longitudinal type: after the homogenization, the two different soap masses, which can differ by virtue of the composition of the soap mass itself or else by virtue of at least one of the abovementioned additives, are extruded by coextrusion (FIG. 8, step 4) to give a soap slug.

Alternatively, various liquid additives can also be added to the soap mass by metered addition via nozzles at a later point in time (FIG. 8, step 3).

For this, the two soap masses are first preshaped in individual e.g. vertically semicircular, extrusion pipes to give partial soap slugs, brought together in a common e.g. circular, extrusion pipe and then extruded through a mouthpiece (FIGS. 9A, 9B). This produces a vertically longitudinally divided soap slug in which the soap phases have already been joined together by the coextrusion.

Alternatively, by rotating the semicircular extrusion pipes for the partial soap slugs or by rotating the separating wall between the partial soap slugs from the perpendicular by an angle between 20° to 70° with respect to the longitudinal axis, a soap slug rotated from the perpendicular with respect to the two soap phases is obtained.

It is preferable if the separating wall between the two individual extrusion pipes is a straight wall. It is also preferred if the separating wall has a wave shape matched to the subsequent soap shape, or has an irregular shape.

It is preferred if the individual extrusion pipes are semicircular and the coextrusion pipe and the mouthpiece (FIGS. 9A, 9B) are circular in their geometric shape. Alternatively, the individual extrusion pipes may be semi-oval and the coextrusion pipe and the mouthpiece may be oval. It is also preferred if the individual extrusion pipes are square or rectangular and the coextrusion pipe and the mouthpiece are rectangular or square. Further combinations of circular, semicircular and angular extrusion pipes are possible.

Various jacket temperatures can be used during the extrusion depending on the extruder used or the screw press used and the soap masses.

In the case of a vertically divided soap slug, the soap slug is firstly cut at an angle of 90° to give the individual soap slugs and then, before or during the stamping of the soap bar, rotated by an angle of from 20° to 70° with respect to the longitudinal axis of the soap slug.

The stamping of the cut and combined soap slugs to give the soap bars is usually carried out individually in rotating disc presses or else in greater numbers in so-called flashstamping systems (Soaps and Detergents, Luis Spitz, pp. 193-204, ISBN 0-935315-72-1).

The stamping operation is carried out with a varying pressing weight or pressing force depending on the type of soap stamping machine used. Various temperatures are used during the stamping operation, corresponding to the nature and composition of the soap mass.

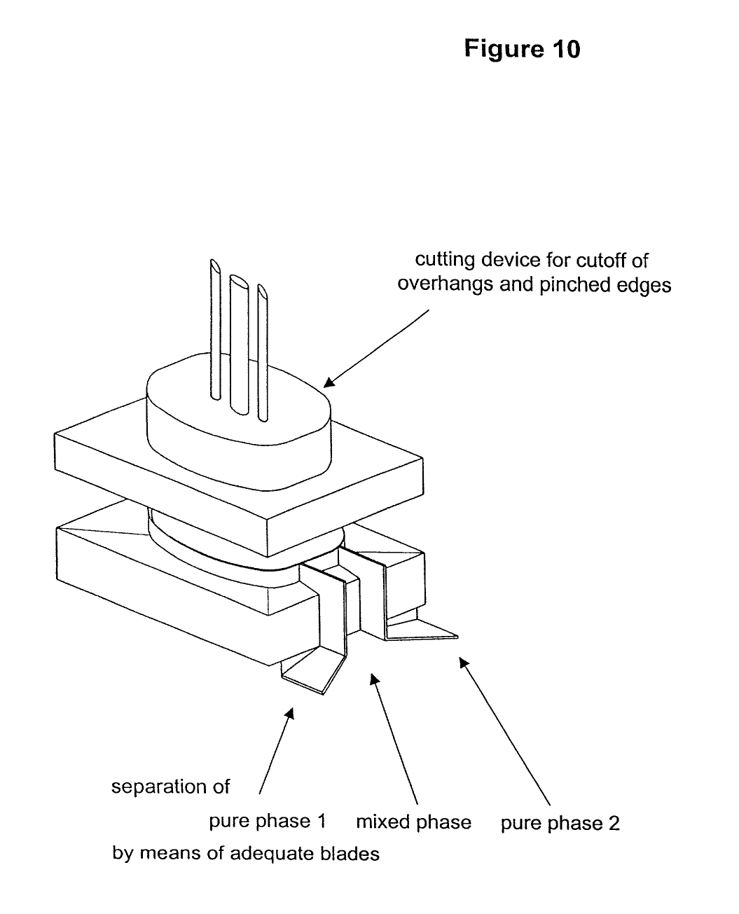

The overhangs or pinched edges which arise during stamping are separated from the soap bar using blades or guides (FIG. 10) during the actual stamping operation. Here, by virtue of a suitable arrangement of the blades, both the two pure soap masses and also a mixed phase is separated off. The two pure soap phases are then returned to the preparation process. For the die according to FIG. 10, a mixed phase of about 1.5%, based on the total soap mass used in the first substep, arises, depending on the angle used.

This mixed phase can, for example, either be processed to give a separate soap or, in the case of the two-colored soap, can, for example, be colored and likewise reused.

Finally, the resulting soap bars are cooled to normal temperature in cooling systems.

Formulations of Various Types of Soap:

For the preparation of multiphase soaps according to the preparation process of the invention, it is possible to use, as soap bases, e.g. alkali soaps, syndets or combinations of the two.

For all of the combinations of the basic soap masses, the water content of the individual soap formulations must be taken into consideration. The varying shrinkage of the individual soap formulations may lead to separation at the contact surface and thus to breakage of the soap. In addition, different soap formulations can have differing wearing-away behavior. By suitably setting the water content, the soap raw materials and further additives in the individual soap formulations and by using the preparation process according to the present invention, it is possible to use numerous combinations of soap formulations for the preparation of stable multiphase soaps.

Since these so-called solid skin cleansing agents can also be prepared in transparent or opaque form on the basis of different additives and a particular preparation process, the most diverse combinations, including of course colored ones, can be prepared.

Reference Numeral List:

FIGS. 1A and 1B

Two-phase soap of the transverse type

Ref. No. 1-phase 1

Ref. No. 2-phase 2

FIGS. 2A and 2B

Multiphase soap with different cutting angles

(transverse type, middle section, side and top view)

FIGS. 3A and 3B

Two-phase soap of the longitudinal type

Ref. No. 1-phase 1

Ref. No. 2-phase 2

FIGS. 4A and 4B

Multiphase soap with different cutting angles

(longitudinal type, middle section, side and top view)

FIG. 5

Schematic preparation process according to process A for the transverse type

FIG. 6

Cutting machine with conveyor belts

FIG. 7

Dies for targeted pinched edge removal and pinched edge separation for the transverse type

FIG. 8

Schematic preparation process according to process B for the longitudinal type and the transverse type

FIGS. 9A and 9N

Extrusion system for processes B and C

FIG. 10

Dies with targeted pinched edge removal and pinched edge separation for the longitudinal type

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A continuous process for the preparation of multiphase soaps comprising two or more separately extruded phases, comprising the step of stamping of multiphase soap slugs which results in mutual cambering, said finished multiphase soap having high stability and the individual separate phases of the resulting multiphase soap are readily visible when viewed from above and from the side.

2. A continuous process according to claim 1, wherein after the steps of milling, homogenization or kneading and the simultaneous extrusion of two or more different soap masses to give soap slugs,
a) the soap slugs are cut in a diagonal direction at an angle of from 20° to 70° relative to the longitudinal direction of the soap slug;
b) bringing together the billets from the soap slugs to give one soap slug;
c) cutting the combined soap slug in a perpendicular direction at an angle of 90° relative to the longitudinal direction of the soap slug; and
d) joining the diagonal points of intersection under pressure so that cambering arises at the points of intersection.

3. A continuous process according to claim 1, wherein after the steps of milling, homogenization or kneading and the simultaneous extrusion of two or more different soap masses to give soap slugs,
a) cutting the two soap slugs in a diagonal direction at an angle of from 20° to 70° relative to the longitudinal direction of the soap slug;
b) cuffing the combined soap slug in the perpendicular direction at an angle of 90° relative to the longitudinal direction of the soap slug;
c) mutually bringing together of the billets from the two soap slugs to give soap slugs; and
d) joining the diagonal points of intersection under pressure so that cambering arises at the points of intersection.

4. A continuous process according to claim 1, wherein after the steps of milling, homogenization or kneading and the simultaneous extrusion of two different soap masses to give soap slugs,
a) cutting the two soap slugs in the perpendicular direction at an angle of 90° relative to the longitudinal direction of the soap slug;
b) cutting the two soap slugs in a diagonal direction at an angle of from 20° to 70° relative to the longitudinal direction of the soap slug;
c) mutually bringing together the billets from the two soap slugs to give soap slugs,
d) joining the diagonal points of intersection under pressure so that cambering arises at the points of intersection.

5. A continuous process according to claim 1, wherein after the steps of milling, homogenization or kneading and the simultaneous extrusion of two or more different soap masses to give soap slugs,
a) coextruding the two soap slugs from different soap phases with vertical contact surfaces through a mouthpiece;
b) cutting the two joined soap slugs at an angle of 90°;
c) rotating the billets by an angle of from 20° to 70° about the longitudinal axis from the perpendicular; and
d) joining the contact surfaces under pressure so that cambering arises at the points of intersection.

6. A continuous process for the preparation of multiphase soaps according to claim 1, wherein after the steps of milling, homogenization or kneading and the simultaneous extrusion of two different soap masses to give soap slugs,
a) coextruding two soap slugs comprising different soap phases with a contact surface through a mouthpiece, the contact surfaces being rotated by an angle of from 20° to 70° about the longitudinal axis from the perpendicular;
b) cutting the two joined soap slugs at an angle of 90°; and
c) joining the contact surfaces under pressure so that cambering arises at the points of intersection.

7. A continuous preparation process according to claim 1, wherein each phase of the resulting multiphase soaps is visible in the perpendicular, longitudinal and transverse projection to at least 15% based on the total projected area.

8. A continuous preparation process according to claim 1, wherein each phase of the resulting multiphase soaps is visible in the perpendicular, longitudinal and transverse projection to at least 20% based on the total projected area.

9. A continuous preparation process according to claim 1, wherein adjacent phase surfaces of the resulting multiphase soaps have been cut diagonally and cambered towards one another.

10. A continuous process for the preparation of multiphase soaps according to claim 1, wherein the individual soap slugs are cut straight diagonally at an angle of from 25° to 60°.

11. A continuous process for the preparation of multiphase soaps according to claim 10, wherein in that the individual soap slugs are cut straight diagonally at an angle of from 30° to 50°.

12. A continuous process for the preparation of multiphase soaps according to claim 5, wherein the individual extrusion pipes are semicircular, and the coextrusion pipe and the mouthpiece are circular in their geometric shape.

13. A continuous process for the preparation of multiphase soaps according to claim 6, wherein the individual extrusion pipes are semicircular, and the coextrusion pipe and the mouthpiece are circular in their geometric shape.

14. A continuous process for the preparation of multiphase soaps according to claim 5, wherein the individual extrusion pipes are semioval, and the coextrusion pipe and the mouthpiece are oval in their geometric shape.

15. A continuous process for the preparation of multiphase soaps according to claim 6, wherein the individual extrusion pipes are semioval, and the coextrusion pipe and the mouthpiece are oval in their geometric shape.

16. A continuous process for the preparation of multiphase soaps according to claim 5, wherein the individual extrusion pipes are square or rectangular, and the coextrusion pipe and the mouthpiece are rectangular or square.

17. A continuous process for the preparation of multiphase soaps according to claim 6, wherein the individual extrusion pipes are square or rectangular, and the coextrusion pipe and the mouthpiece are rectangular or square.
